(19) United States
(12) Patent Application Publication
(10) Pub. No.: US 2008/0193968 A1
(43) Pub. Date: Aug. 14, 2008

(54) Therapeutic Uses of Artificial Nanostructures

(76) Inventors: Bing Xu, Hong Kong (CN); Zhimou Yang, Hong Kong (CN); Keming Xu, Hong Kong (CN)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/063,079

(22) PCT Filed: Mar. 6, 2006
(86) PCT No.: PCT/US2006/008333

§ 371 Date: Feb. 6, 2008

Related U.S. Application Data
(60) Provisional application No. 60/706,072, filed on Aug. 8, 2005.

(51) Int. Cl. C12P 1/00

(52) U.S. Cl. 435/41

(57) ABSTRACT

The present invention provides a method for controlling the fate of mammalian cells or microorganisms by the enzymatic formation of intracellular nanostructures. Enzymatic reactions trigger the intracellular self-assembly to convert a proper precursor into another molecule or nanoobject that will aggregate inside cells or inside or between tissues or organs. Further, this invention provides a method for making artificial nanostructures inside or between tissues or organs, by injecting a proper designed precursor into tissues or organs, and enzymatic reaction converting the precursor to a hydrogelator to form artificial nanostructures and inducing hydrogelation and at the state of a disease, another enzyme converts the hydrogelator back to precursor to induce gel-to-sol transition to release a drug. The present invention can be applied to treat diseases caused by the malfunction of cells, infection caused by microorganisms and provides a novel route for controlled drug releases, formation of new therapeutic agents, and in-situ formation of hydrogel to treat degenerative diseases such as osteoarthritis.

This application claims the benefit of U.S. Ser. No. 60/706,072, filed Aug. 8, 2005, the contents of which are incorporated herein in its entirety by reference.

Throughout this application, various references are cited and disclosures of these publications in their entireties are hereby incorporated by reference into this application in order to more fully describe the state of the art to which this invention pertains.

### BACKGROUND OF THE INVENTION

This invention is directed to create intracellular artificial nanostructures inside cells or microorganisms. Such artificial nanostructures can be applied for controlling the fate of the cells or microorganisms selectively, but not limited to the above, for treating diseases that are caused by pathogenic cells or microorganisms. In addition, the same strategy is used for generating artificial nanostructures inside or between tissues or organs for therapeutic benefits.

Self-assembly, a fundamental process at all scales, plays a vital role in biology and provides an important guidance for the design and fabrication of functional materials. Particularly, self-assembly provides an attractive and practical methodology for creating artificial nanostructures that promise broad impacts and applications in the emerging field of nanoscience: for examples, self-assembled nanoparticles may lead to novel optical materials and high-density magnetic recording media; the self-assembled monolayers have enabled nanometer thickness organic films to be constructed on a variety of substrates for modeling biological surface to control the fate of cells, building molecular electronic devices, developing nanolithography, and generating nanostructures for biomedical diagnostics. The self-assembly of oligopeptides and other organic molecules has resulted in nanofibers as the functional matrices of hydrogels that are useful for tissue engineering, inhibitor screening, and wound healing. Although these works reflect exciting and important development of self-assembled nanostructures in extracellular settings or a non-biological arena, intracellular creation of artificial nanostructures remains unexplored and its subsequent biological effects unknown despite of its significances and potential applications.

Exploring intracellular artificial nanostructures is significant for several reasons. First, self-assembled nanostructures such as cell membranes, strands of nucleic acids, and actin filaments, prevail in living cells and are indispensable for critical cellular functions (i.e., as structural motifs for maintaining integrity of cells, as effective storages for keeping genetic information; and as active devices for regulating numerous of cellular processes), therefore intracellular artificial nanostructures provide an attractive and effective strategy from perturbing the cellular activities to managing the behaviors of cells. Second, many diseases are related to mishaps in cellular nanostructures (i.e., mismatch of base pairs, formation of β-amyloid, and misfolding of proteins), hence intracellular artificial nanostructures offers a versatile platform for mimicking, modeling, and understanding the mechanism of diseases, thus developing the therapeutic approaches. Third, spectacular advances in molecular cell biology such as the study of biological process at the molecular level, during the last five decades have led to new insights into the evolution of life form, and now there is a need to correlate biological process beyond molecule level and to understand structure and dynamics as a system (i.e., system biology). Self-assembled intracellular artificial structures at nanoscale lend a convenient means to examine the structure and dynamics of cellular and organismal function and to allow previously unconnected domains of knowledge to be understood at new levels of complexity.

This present invention also reports a general strategy to control the states of an artificial nanostructure that induces hydrogelation via an enzymatic switch¹ and in vivo hydrogelation. As one of the most utilized biomaterials in drug delivery, wound healing, tissue engineering, hydrogels usually use natural or synthetic polymers as the gelators.² Largely because of the study of low molecular weight organogelators³ and the demonstration of hydrogels made of self-assembled oligopeptides as scaffolds for tissue engineering,⁴⁻⁶ the range of hydrogelators has expanded rapidly to include small molecules, which make possible supramolecular hydrogels, in the past decade.⁷⁻⁹ The self-assembly of the hydrogelators plays a key role during the formation of nanostructures in a supramolecular hydrogel.⁷ Therefore, triggering or regulating the self-assembly of hydrogelators becomes an essential step in controlling the states and properties of supramolecular hydrogels, which is normally achieved by chemical or physical perturbations (i.e., pH, temperature, ionic strength, and ultrasonic agitation). In biomedical applications, enzyme-catalyzed, in-situ, including both extra- (inside or between tissue or organs) and intracellular reversible self-assembly and gelation of the hydrogelators is advantageous¹⁰ because it allows the hydrogels to respond to the expressions of specific enzymes for certain tissues, organs, or diseases. Despite the use of an enzyme to cross-link polymers to induce hydrogelation¹¹ and reports on an enzyme-triggered formation of supramolecular hydrogels,⁹ the use of enzymes to regulate supramolecular hydrogels (i.e., for reversible control of the self-assembly of the hydrogelators) has yet to be explored.

Because most enzymatic reactions are essentially irreversible, a single enzyme hardly ever controls hydrogels in a reversible manner. Nature solves a similar dilemma by using a pair of enzymes that have counteracting activities to switch the functions of proteins.¹ This invention mimics nature by using a kinase/phosphatase switch to regulate supramolecular hydrogels.

As illustrated in Scheme 1, two types of enzymatic reactions can lead to the formation of hydrogelators (IV): (A) Enzyme catalyzed bond-cleavage which restores the balance of hydrophobicity and hydrophilicity of a hydrogelator; and (B) Enzyme catalyzed bond formation which balances hydrophobicity and hydrophilicity on a hydrogelator. After enzymatic formation, the hydrogelators (IV) will self-assemble in water to afford supramolecular polymers, which further aggregate to form a nanofibers network as the matrices for the resulting hydrogels. Additionally, in biological systems, one of most common mechanisms for regulating the activity of a protein is an enzymatic “switch”, of which two enzymes have counteracting activities (i.e., one enzyme catalyzes the formation of a specific bond, and the other catalyzes the cleavage of the bond). An enzymatic switch such as the one described above, can also regulate the process of the hydrogelation of a small molecule hydrogelator, which would create hydrogels that respond to the change of a biological environment. This is elaborated in the following section.

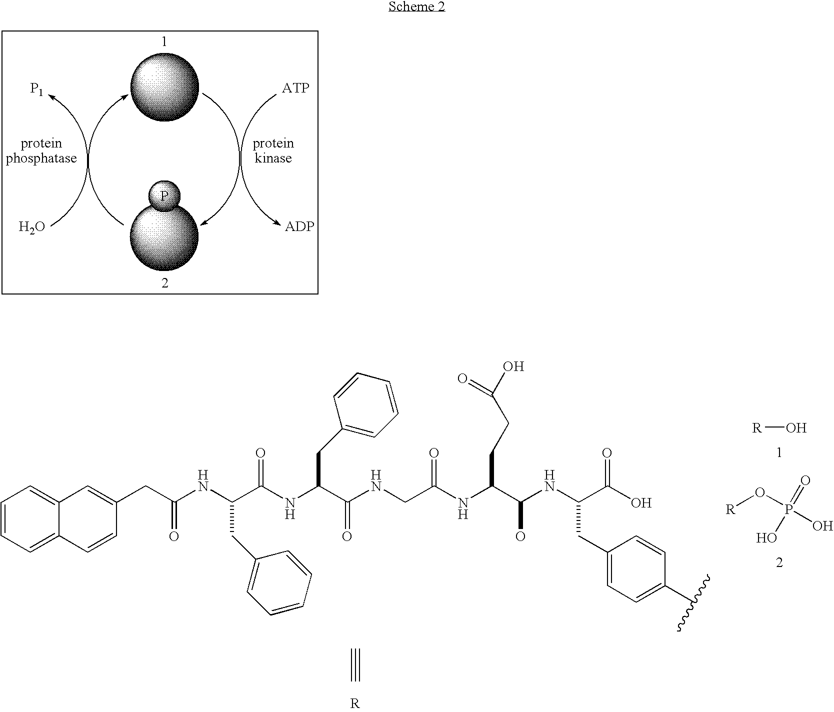

As shown in Scheme 2, a pentapeptidic hydrogelator was synthesized, Nap-FFGEY (1), which forms hydrogels at 0.6 wt % via the self-assembly of 1. Adding a kinase to the hydrogel in the presence of adenosine triphosphates (ATP) phosphorylates 1 to give the corresponding phosphate (2), thus disrupting the self-assembly to induce a gel-sol phase transition; treating the resulting solution with a phosphatase dephosphorylates 2 to form 1, thus restoring the self-assembly to form the hydrogel. Moreover, subcutaneous injection of 2 in mice leads to the formation of supramolecular hydrogel in vivo.

In addition to being the first demonstration of an enzyme-switch-regulated supramolecular hydrogel and the formation of supramolecular hydrogels in vivo by an enzymatic reaction, the combination of the kinase/phosphatase switch with supramolecular hydrogels promises a new way to make and apply biomaterials because phosphorylation and dephosphorylation, as common yet important biological reactions, occur in many organisms. Since many diseases such as cancer,¹² diabetes,¹³ Alzheimer's disease,¹⁴ and multiple sclerosis¹⁵ are associated with the abnormal activities of phosphatases and/or kinases,¹⁶ enzyme-switch-regulated hydrogelation, compared with conventional physical and chemical processes, should be superior because it enhances the biologically specific response of the hydrogels regarding the level of enzyme expression.

Furthermore, investigation of the enzyme-switch-regulated self-assembly of hydrogelators helps to create an understanding of the functions of supramolecular hydrogels in a biological environment where multiple enzymes exist.

### SUMMARY OF THE INVENTION

The present invention pertains to (1) the design and application of a new type method of making artificial nanostructures inside cell or microorganisms, comprising of the steps of: (i) constructing a proper design precursor which does not self-assemble extracellularly, with said cells or microorganisms under conditions permitting the entrance of the precursor into said cells or microorganisms; (ii)placing the cells or microorganisms under conditions permitting the precursor into nanostructures; (2) the design and application of a new type of method of making artificial nanostructures inside or between tissues or organs, comprising of the steps of, injecting a proper designed precursor into tissues or organs, and an enzymatic reaction converting the precursor to a hydrogelator to form artificial nanostructures and inducing hydrogelation; and (3) the design and application of a new type of method of making artificial nanostructures inside or between tissues or organs, comprising of the steps of, injecting a proper designed precursor and therapeutic agents into tissues or organs, and an enzymatic reaction converting the precursor to a hydrogelator to form artificial nanostructures and inducing hydrogelation, and at the state of a disease, another enzyme converts the hydrogelator back to precursor to induce gel-to-sol transition to release a drug.

The present invention provides a general method for control the fate of mammalian cells or microorganisms by the formation of intracellular nanostructures via an enzymatic reaction or enzymatic reactions. The formation of intracellular nanostructures, for example nanofibers or aggregates of nanoobjects, is accomplished by intracellular self-assembly of molecules or nanoobjects. The intracellular self-assembly is triggered by using enzymatic reactions to convert a proper precursor, for example, molecules or nanoobjects, into another molecule or nanoobject that will aggregate inside cells or inside tissues or organs. Such intracellular nanostructures can cause the hydrogelation of water inside cells or the interruption the endogenous cellular activities, which can change the behavior of cells or microorganisms or organisms. For example, such intracellular nanostructures can be used to trigger the death of cells or microorganisms or organisms, to stop the differentiation of cells including stem cells or to inhibit the growth of microorganisms or organisms, and to change the differentiation behaviors of cells, including stem cells, or organisms. The creation of such intracellular nanostructures can be specific to pathogenic cells or microorganisms, thus the method of present invention can be applied to treat diseases caused by the malfunction of cells or infection caused by microorganisms or organisms.

In addition, inside or between the tissues or organs, uses of enzymatic reaction convert the precursor to a hydrogelator to form artificial nanostructures and induce hydrogelation and at the state of a disease, another enzyme converts the hydrogelator back to precursor to induce gel-to-sol transition to release a drug. This approach provides a novel route for controlled drug releases, formation of new therapeutic agents, and in-situ formation of hydrogels to treat chronic diseases, such as osteoarthritis.

### DESCRIPTION OF THE FIGURES

FIG. 1. (A) Schematic intracellular nanofibers formation that lead to hydrogelation and cell death. (B) The chemical structures and graphic representations of a precursor molecule (1a) and its corresponding hydrogelator (2a).

FIG. 2. (A) Oscillatory rheology of a solution containing 8 mM (0.5 wt %) of 1a and 0.2 mg of enzyme solution, pH=8.0, 37° C.; (B) TEM images of the hydrogels (inset: optical image) formed by 1a via enzymatic gelation in water (conc.=0.5 wt %, pH=8.0); (C) UV spectra of the cell culture media (DMEM), the gel in FIG. 2B, and the HeLa cells before and after culturing with 1a; and (D) TEM of the hydrogels (inset: optical image) formed by the dead Hela cells after culturing with 1a for three days (arrows indicated the nanofibers formed by 2a).

FIG. 3. Optical images (×100) of the growth of HeLa cells and NIH3T3 cells after being cultured in the media containing 0.02, 0.04, and 0.08 wt % of 1a in 0, 1, 2, and 3 days. The focal plane of microscope was adjusted on the bottom of the petri dish, the shape change and the decrease of the cells on the surface indicated cell death.

FIG. 4. Optical images and corresponding HPLC traces of (A) gel I; (B) the solution obtained after adding a kinase to gel I; and (C) gel II. The hydrogels are on the right side of the panels (A, C), viewing from the bottoms of the vials. The solution containing 1 and 2 in almost 1:1 ratio forms a meniscus in the tilted vial (panel B).

FIG. 5. (A) Dynamic strain sweep of gel I at the frequency of 1.0 rad/s; (B) dynamic frequency sweep of gel I at the strain of 0.8%; and (C) dynamic time sweep of the solution containing 0.3 wt % of 1, 0.3 wt % of 2, and 400 U/mL of alkali phosphatase in the buffer at the strain of 0.8% and the frequency of 1 rad/s.

FIG. 6. TEM images of the cryo-dried (A) gel I; (B) gel II; (C) solution of 1 (0.3 wt %) in the buffer; and (D) solution containing 1 (0.32 wt %) and 2 (0.28 wt %) in the buffer.

FIG. 7. (A) Circular dichroism spectra of gels I and II and (B) fluorescent spectra (λₑₓ=272 nm) of solution of 1, gel I, and gel II.

FIG. 8. One of the possible molecular arrangements of 1 in nanotubes of gel II: the, β-sheet features due to hydrogen bonding (A); the molecular organization of 1 along (B) and crossing to (C) the nanotubes; and the overall molecular packing in the nanotubes (D).

FIG. 9. MTT assay on the HeLa cells treated by 1 and 2.

FIG. 10. (A) Optical image of the hydrogel formed subcutaneously 1 h after injecting 2 into the mice; (B) the optical image of the abdominal cavity of a mouse 1 h after the injection of 2; (C) HPLC trace of the hydrogel shown in FIG. 10A; and (D) HPLC trace of the abdominal fluid 1 h after injecting 2.

FIG. 11. Weight gain of the mice (n=6, initial body weight=20±2 g) after (A) subcutaneously and (B) intraperitoneally injecting 0.5 mL of 2 at 0.8 wt % concentration. Saline solution (0.5 mL) served as the controls for both modes of injection.

### DETAILED DESCRIPTION OF THE INVENTION

The present invention pertains to (1) the design and application of a new type method of making artificial nanostructures inside cell or microorganisms, comprising of the steps of: (i) constructing a proper design precursor which does not self-assemble extracellularly, (ii) contacting said precursor with said cells or microorganisms under conditions permitting the entrance of the precursor into said cells or microorganisms; (iii) placing the cells or microorganisms under conditions permitting the precursor into nanostructures; and (2) the design and application of a new type method of making artificial nanostructures inside tissues or organs, comprising of the steps of, injecting a proper designed precursor into or between tissues or organs, and enzymatic reaction converting the precursor to a hydrogelator to form artificial nanostructures and inducing hydrogelation; and (3) the design and application of a new type of method of making artificial nanostructures inside or between tissues or organs, comprising of the steps of, injecting a proper designed precursor and therapeutic agents into tissues or organs, and an enzymatic reaction converting the precursor to a hydrogelator to form artificial nanostructures and inducing hydrogelation, and at the state of a disease, another enzyme converts the hydrogelator back to precursor to induce gel-to-sol transition to release a drug.

The present invention for making artificial nanostructures intracellularly or inside or between tissues or organs, which can be specific to pathogenic cells or microorganisms, through, but not limited to, an enzymatic reaction triggered supramolecular hydrogelation comprising of the steps of: (1) constructing a proper design precursor which does not self-assemble extracellularly, which enters the cell by, but not limited to, a simple diffusion process; (2) an enzyme expressed in the cell converts the precursor into a hydrogelator that can self-assembled into nanofibers which is one of simple nanostructures; and (3) the formation of nanofibers can lead to hydrogelation, which exerts stresses on the cell, and causes cell death, which is an easily observable cellular transition.

Such nanostructures created intracellularly or inside or between tissues or organs, can also be used to stop the differentiation of cells including stem cells or to inhibit the growth of microorganisms and to change the differentiation behaviors of cells, including stem cells, or organisms.

The method of the present invention can be applied to treat diseases in both animals and humans caused by the malfunction of cells or infection caused by microorganism because the creation of such nanostructures, intracellularly or inside or between tissues or organs, can be specific to pathogenic cells or microorganisms.

An effective amount of the precursor of the present invention can be administered to treat diseases such as cancer, diabetes, Alzheimer's disease, or multiple sclerosis.

The basic structure of the precusor for enzymatic hydrogelation is: (1) a hydrophobic group (i.e., napthyl, aromatic, hydrocarbon tails-either linear or branched; (2) a hydrophilic group (i.e., di-, tri-, tretra-, penta, or oligopeptides soluble in water, carboxylate, aminoglycosides, antibiotics, other water-soluble therapeutics); and (3) a cleavable group (i.e., phosphate, butryic acid, sulfate, ammonium, ethylene glycol).

The method of the present invention consists of a proper design precursor which does not self-assemble extracellularly and is synthesized consisting of three distinct motif: (1) a group, including napthyl (C₁₀H₇CH₂—), alkyl (CnH2n+1, n=4-30), and aromatic groups, for providing the hydrophobic force to enhance the self-assembly in aqueous environment; (2) the molecular or nanoscale segment (i.e., single amino acid residues, dipeptide, phe-phe or X—Y, X, Y are amino acid residues, and tripeptides, tetrapeptides, pentapetides, aminoglycosides, fluoroquinolones, bisphosphonates, antibiotics, antineoplastic, antifungual, antiparasitic molecules, iron oxide nanoparticles (5 to 50 nm), etc.) being the major building blocks for self-assembly besides acting as hydrogen bonds acceptors and donors to interact with water; and (3) a cleavable group (i.e., butyric dicarboxylate acid, bisphosphonates, phosphates, carbohydrates, etc.), which covalently links to the segment by a link (covalent or noncovalent) that can be broken by an enzyme or enzymatic switch for tailoring the overall balance of the hydrophobic and hydrophilic interactions and to prevent the hydrogelation of the precursor without the enzymatic reaction.

This invention further provides the design and application of a new type method of making artificial nanostructures inside or between tissues or organs, comprising of the steps of, injecting a proper design precursor into tissues or organs, and an enzymatic reaction which converts the precursor to a hydrogelator to form artificial nanostructures and induces hydrogelation and at the state of a disease, another enzyme converts the hydrogelator back to precursor to induce gel-to-sol transition to release a drug.

Inside or between the tissues or organs, uses of enzymatic reaction convert the precursor to a hydrogelator to form artificial nanostructures and induce hydrogelation and at the state of a disease, another enzyme converts the hydrogelator back to precursor to induce gel-to-sol transition to release a drug. This approach provides a novel route for controlled drug releases, formation of new therapeutic agents, and in-situ formation of hydrogels to treat chronic diseases, such as osteoarthritis.

This invention also provides a method for treating a subject with cancer, diabetes, Alzheimer's disease, or multiple sclerosis, chronic diseases such as osteoarthritis, comprising administering to said subject an effective amount of precursor which is capable of forming nanostructures intracellularly or inside or between tissues or organs. As it can be appreciated by and ordinary skilled artisan, this invention may be used to treat or prevent other diseases. In an embodiment, the precursor is a nap-FFGEY type precursor.

As used herein, the subjects include mammal. In an embodiment, the subjects are animals. In another embodiment the subjects are humans.

The invention also provides different uses of the constructed precursor for treatment and prevention of diseases. Finally, the invention provides kits which contain the precursor described supra.

The invention will be better understood by reference to the Examples which follow, but those skilled in the art will readily appreciate that the specific examples are only illustrative and are not meant to limit the invention as described herein, which is defined by the claims which follow thereafter.

### EXPERIMENTAL DETAILS

### Example 1

### Creation of Intracellular Nanostructures

To illustrate the general concept of creating Intracellular nanostructures an enzymatic reaction triggered supramolecular hydrogelation in a cell was used.

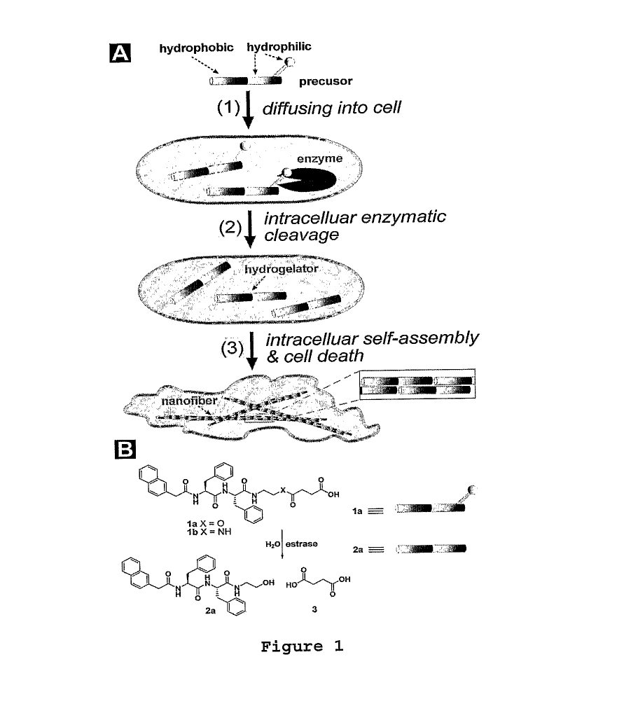

Intracellular nanostructures may be created using different methodologies, which should share several common features: (i) the building blocks must self-assemble to give nanostructures; (ii) the self-assembled nanostructures should be exclusively formed inside cells; (iii) the self-assembly should be initiated by or coupled to an intracellular process; and (iv) the formation of nanostructures can lead to observable phenomena or cellular transitions for easy identification. As shown in FIG. 1A, a proper design precursor which does not self-assemble extracellularly, enters the cell by, but not limited to, a simple diffusion process; once the precursor is inside the cell, an enzyme expressed in the cell converts the precursor into a hydrogelator that can self-assembled into nanofibers which is one of simple nanostructures; the formation of nanofibers can lead to hydrogelation, which exerts stresses on the cell, and cause cell death which is an easily observable cellular transition.

To satisfy the concept illustrated in FIG. 1A, a precursor was designed and synthesized (1a, FIG. 1B) to consist of three distinct motif: a group, including napthyl (C₁₀H₇CH₂—), alkyl (CnH2n+1, n=4-30), and aromatic groups, for providing the hydrophobic force to enhance the self-assembly in aqueous environment; (i.e., single amino acid residues, dipeptide, phe-phe or X—Y, X, Y are amino acid residues, and tripeptides, tetrapeptides, pentapetides, aminoglycosides, fluoroquinolones, bisphosphonates, antibiotics, antineoplastic, antifungual, antiparasitic molecules, iron oxide nanoparticles (5 to 50 nm), etc.) being the major building blocks for self-assembly besides acting as hydrogen bonds acceptors and donors to interact with water; and (3) a cleavable group (i.e., butyric dicarboxylate acid, bisphosphonates, phosphates, carbohydrates, etc.), which covalently links to the segment by a link (covalent or noncovalent) that can be broken by an enzyme or enzymatic switch for tailoring the overall balance of the hydrophobic and hydrophilic interactions and to prevent the hydrogelation of 1a without the enzymatic reaction.

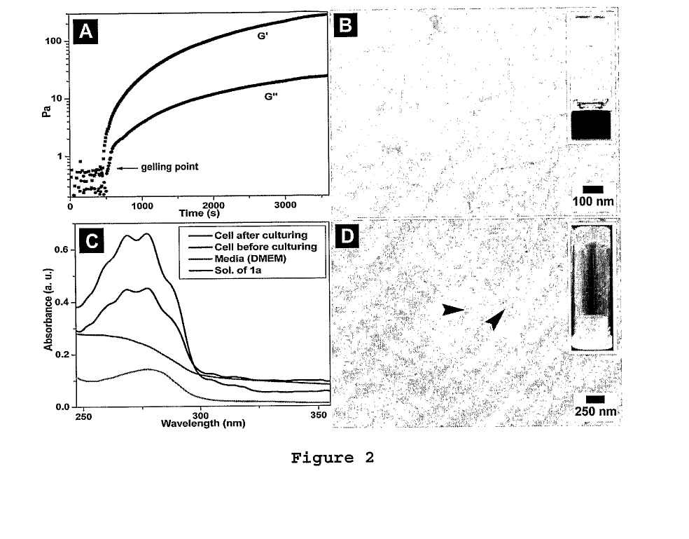

To characterize the properties of 1a, the fact that an esterase can convert 1a to 2a, lead to the formation of nanofibers, and induce hydrogelation was verified. At pH˜8.0, adding 0.1 mL of the esterase (7 U of esterase in 1 mL of distilled water with pH adjusted to 8.0) to 0.9 mL solution of 1a (0.5 mg) and keeping the solution at 37° C. for about 6 minutes resulted in the formation of the hydrogel, which is stable even upon heating to near 100° C. Rheological experiments (FIG. 2A) reveal that the hydrogel starts to form in less than 10 minutes, as indicated by the storage modulus (G′) dominating the loss modulus (G″). This enzyme-catalyzed hydrogelation completes in 100 minutes, as indicated by the storage modulus (G′) reaching the plateau. ¹H NMR suggests that 68% of 1a transforms to 2a at this stage. The formed hydrogel of 2a is transparent (inset, FIG. 2B), suggesting that there is no microcrystalline aggregate in the hydrogel to scattering visible light, which agrees with the transmission electron micrograph (TEM) of the hydrogel (FIG. 2B). In addition, TEM shows that the size of the nanofibers formed by the self-assembly of 2a is ˜10 nm, though the bundles of the nanofibers reach the size as wide as ˜60 nm.

After proving the conversion of 1a to 2a by the esterase and the hydrogelation of 2a, the characteristic absorption peaks of the naphthyl group in the culture solution and the Hela cells were monitored to estimate the amount of the precursor uptaken by the cells. After culture with Hela cells had proceeded for three days (in a culture initially containing 0.08 wt % of 1a), the absorption of the naphthyl group dropped 32% in the culture solution (supporting information). Concurrently, the absorption of the naphthyl appeared on the Hela cells. The shape and position of the absorption peak is identical to the absorption spectra of the hydrogel formed by the conversion of 1a to 2a using esterase, suggesting intracellular hydrogelation. Moreover, since the volume of the cells is less than 1% of the volume of the culture media, the concentration of 1a inside the cells easily reaches above the mgc of 2a. Once these molecules of 1a are converted to 2a by endogenous esterases, it should self-assemble into nanofibers. To confirm this, the dead Hela cells that are detached from the surface of the culture solution were collected. After using centrifuge to remove the extracellular water, the cells were broken and observed the formation of hydrogel (inset, FIG. 2D). TEM (FIG. 2D) reveals that the formation of nanofibers with the width of 25 nm and morphology similar to the nanofibers formed by the 2a alone. Live Hela cells were collected that adhered to the surface of petri dish. After being broken by ultrasound, the cell debris neither forms hydrogel nor shows long nanofibers under TEM. These results confirm that the cell death is associated with intracellular formation of the nanofibers and the hydrogelation.

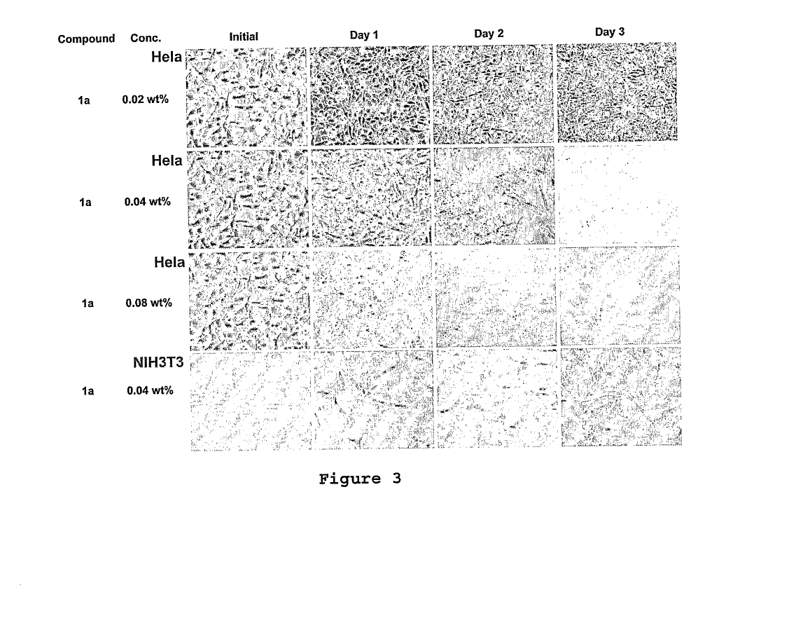

After confirming that the hydrogelation can proceed as designed inside the cells, the precursor 1a was tested in a series of concentration to examine its effective concentration required for cell death (FIG. 3). At [1a]=0.02 wt %, the cell growth appears normal. At [1a]=0.04 wt %, although the number of the cell increases in the first day, cell death starts in day two, as evidenced by the decrease of the cell that attached to the bottom of the culture dish; on the day three, almost no cells are alive. At [1a]=0.08 wt %, the number of the cells decreases in day one; in day two, much fewer cells attaches to the surface compared to [1a]=0.04 wt %; and in day three, almost all the cell rounds up and detaches from the surface, indicating cell death. The gradually death of cells agrees with the concentration build-up of 2a, which is necessary for the formation of the nanofibers and hydrogelation. This phenomenon also indicates that the target of 2a is the cytosol water. To further verify that the esterase substrate is selective to Hela cells, 1a was cultured with fibril blast cells (NIH3T3). As shown in FIG. 3, at [1a]=0.04 wt %, the cells continue to multiply, which confirms that 1a is innocuous to normal cell lines.

### Example 2

### Design and Synthesis of a Kinase/Phosphatase Switch to Regulate a Supermolecular Hydrogel and Forming the Supermolecular Hydrogel in Vivo

The Nap-FFGEY (1, Scheme 2) was designed as both the kinase substrate and the hydrogelator because (i) FF is prone to self-assembly,¹⁷ (ii) Nap-FF gels water effectively (at 0.8 wt %¹⁸), and (iii) the residue of Glu-Tyr (EY) accepts phosphorylation in the presence of a tyrosine kinase¹⁹.

One of the motivations to use naphthalene (Nap) rather than N-(fluorenyl-methoxycarbonyl) (FMOC) is that Nap should be more biocompatible, as evidenced by several clinical drugs consisting of a Nap motif (i.e., propranolol, naphazoline, nafronyl). The glycine (G) was used to connect Nap-FF with EY because glycine is the simplest amino acid. Unlike other pentapeptides,⁴ FFGEY is not a known epitope of any protein, but it carries the basic structural requirement to serve as the substrate of the tyrosine kinase. After obtaining 1 through solid-phase synthesis,²⁰ the hydrogelation ability of 1 was tested. Via a slight adjustment of pH (from 7.8 to 7.5), 1 forms a transparent hydrogel in water at 0.6 wt %.²⁰ The successful hydrogelation of 1 implies that Nap-FF also may act as a useful motif to conjugate with other amino acid residues to construct hydrogelators.

### Example 3

### Enzyme Switch Controls the Phase Transition of the Hydrogel

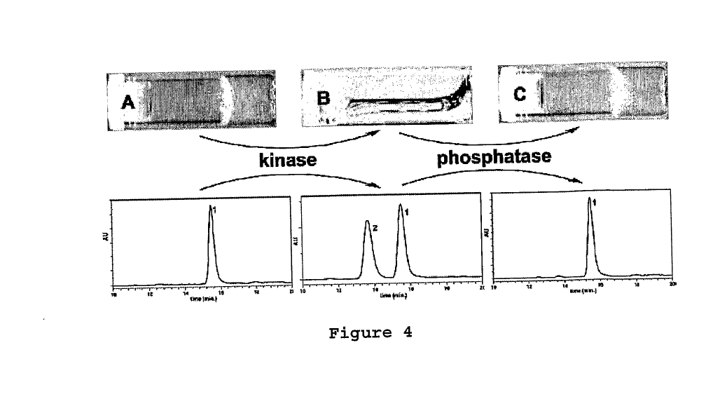

After confirming that 1 is indeed an efficient hydrogelator, the use of the kinase/phosphatase switch to control the phase transition of the hydrogel was examined. The addition of 1 (3 mg) into a buffer (0.5 mL, containing 10 mM of ATP) creates a transparent hydrogel (gel I, FIG. 4A) in 5 min. Then, 3 U of tyrosine kinase (50 μL) was added on the top of gel I to initiate the phosphorylation of 1. After 24 h, gel I turned into a clear solution (FIG. 4B). An HPLC test of the solution confirmed that ˜46% of 1 was converted to 2. Because the phosphate groups of 2 repel each other to weaken the selfassembly of the nanofiber and render 2 more hydrophilic than 1, the gel-sol phase transition occurs. The addition of ˜200 U of alkali phosphatase (10 μL) into the solution restores the hydrogel (gel II, FIG. 4C) in 1 h. After another 4 h, HPLC analysis showed that 99.1% of 2 transformed back to 1. Because the catalytic activity of the phosphatase used in this experiment is about 1000 times higher than that of kinase, one cycle of the gel-sol-gel transformation was able to be completed. To cycle such a transformation many times, one might need to adjust the relative amounts of a pair of enzymes that have similar activities. Nevertheless, the result demonstrated here validates the concept of the regulation supramolecular hydrogels by an enzyme switch. In addition, the new insight of the dynamic cell signaling suggests that a stimulus tips the protein kinase (PK)/protein phosphatase (PP) balance by simultaneously activating PKs and deactivating PPs.¹⁶ This model implies that it would be easier to cycle the phase transition of the supramolecular hydrogel in vivo using proper hydrogelators as the substrates, which may lead to a drug delivery system that responds to biological activities of tissues.

### Example 4

### Rheological Study

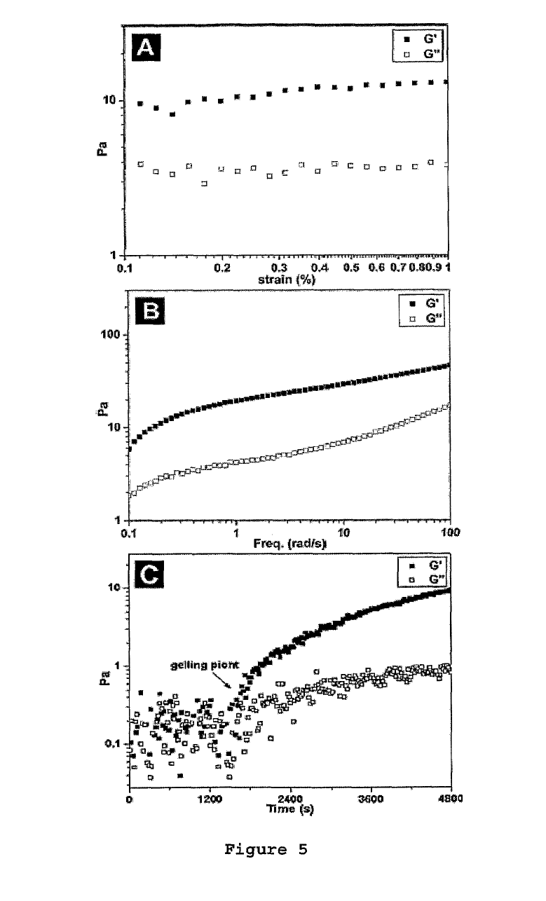

To evaluate the viscoelastic properties of the gel, first dynamic strain sweep was used to determine the proper condition for the dynamic frequency sweep of gel I. As shown in FIG. 5A, the values of the storage modulus (G′) and the loss modulus (G″) exhibit a weak dependence from 0.1 to 1.0% of strain (with G′ dominating G″), indicating that the sample is a hydrogel. After setting the strain amplitude at 0.8% (within the linear response regime of strain amplitude), dynamic frequency sweep was used to study gel I. FIG. 5B exhibits that G′ and G″ slightly increase with the increase of frequency from 0.1 to 100 rad/s. The value of G′ is about five times larger than that of G″ in the whole range (0.1-100 rad/s), suggesting that gel I is fairly tolerant to external force.

To study the enzymatic formation of gel II, the mode of dynamic time sweep was chosen to examine the change of viscoelasticity of the solution containing 1 and 2 in a ratio of 1:1 upon adding the phosphatase. Alkali phosphatase (400 U/mL) was added to the solution of 0.3 wt % of 1 and 0.3 wt % of 2 in the buffer for the rheological measurement. As shown in FIG. 5C, G′ and G″ of the mixture are very small at the moment of the addition of the enzyme, indicating that the solution of 1 and 2 indeed behaves as a low-viscosity liquid when 1 and 2 are in equal amount. Thereafter, both G′ and G″ increase with time, and the value of G′ starts dominating G″ in about 30 min after the addition of the phosphatase, suggesting the approach of the gelling point. After 1.5 h, the plateau value of G′ is about 10 times larger than that of G″, indicating the extensive formation of a three-dimensional matrix in the hydrogel. The time needed to reach the gelling point is shorter than that of a free-standing sample (about 1 h), likely because mechanical perturbations associated with rheological measurements also accelerate the dephosphorylation reaction catalyzed by the enzyme.

### Example 5

### Morphological and Spectral Studies

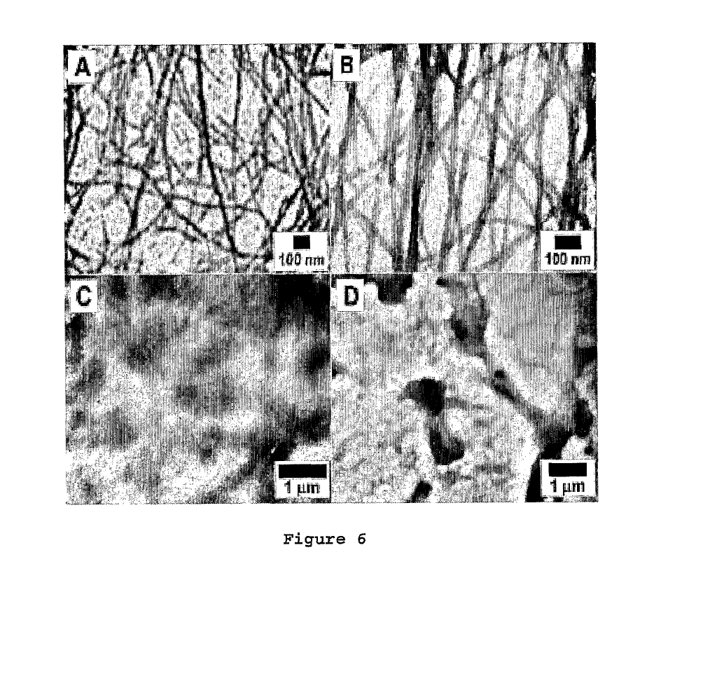

Because they are formed via different processes, gels I and II offer a fine opportunity to evaluate the influence of the enzyme on the selfassembly process. According to the cryo-TEM images of the gels (FIG. 6), 1 self-assembles into nanofibers of various sizes in gel I (with the diameters of 28±5 nm) and into uniform nanotubes in gel II (with diameters of 18±1.5 nm and wall thicknesses of 6 nm), indicating that the enzyme switch regulates the self-assembly process to afford better nanofibers. Two factors likely contribute to the kinetics of self-assembly to give the better defined fibers in FIG. 6B. First, compared to the change of pH to afford gel I in FIG. 6A, the dephosphorylation of 2 by the phosphatase allows the nanofibers to form more slowly, thus permitting a more ordered self-assembled nanostructure. Second, because the enzyme pair catalyzes both phosphorylation and dephosphorylation during the formation of gel II, it is speculated that the tyrosine kinase probably phosphorylates the disorder regions in the nanofibers more easily than it does to the ordered regions. So, the kinase helps to remove the disorder parts by converting 1 to 2, and the phosphatase transforms 2 back to 1 for reassembly. This equilibrium helps to remold the nanofibers into more uniform nanotubes. In that sense, the enzyme switch helps to reject defects in the selfassembly process for forming the nanotubes of 1 in gel II.

To understand the behavior of 1 below minimum gelation concentration and at the stage shown in FIG. 4B, used TEM was also used to examine the morphology of the cryo-dried solution of 1 (0.3 wt %) and the solution containing 1 (0.32 wt %) and 2 (0.28 wt %). As shown in FIGS. 6C and 3D, TEM confirms that there is no extensive formation of nanofibers in both cases except mainly amorphous solids and a small amount of short fibrous structures, suggesting that the concentrations of the hydrogelator in these two conditions are too low to self-assemble into a network of nanofibers for hydrogelation. This result is also consistent with the rheological behavior of the solution containing a 1:1 mixture of 1 and 2.

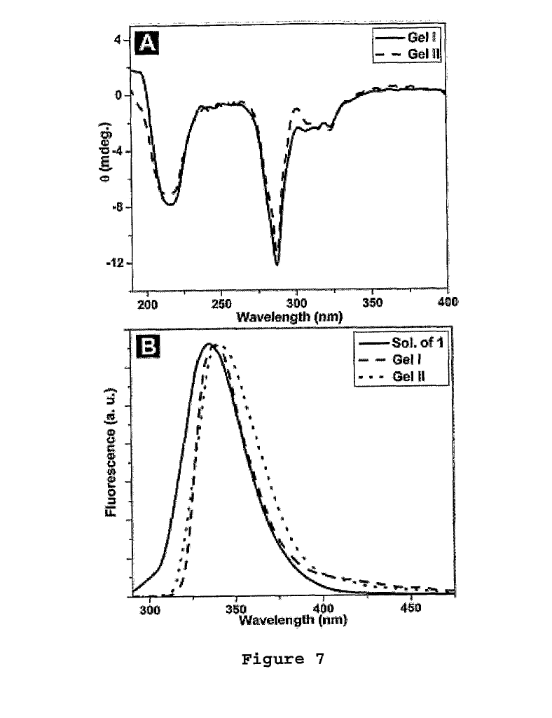

To further understand the molecular arrangement of the hydrogel of 1, measured the circular dichroism and emission spectra of gels I and II were measured. The CD spectra (FIG. 7A) of gels I and II are almost identical, and both exhibit a positive band near 196 nm (ΠΠ* transition), a negative band near 215 nm (nΠ* transition), and a negative band near 287 nm (ΠΠ* of naphthyl aromatics), coinciding with the CD spectra of the nanofibers of oligopeptides⁶ and indicating, β-sheet features. The fluorescent spectra (FIG. 7B) show a peak centered at 338 nm for the solution of 1 and asymmetric peaks with the maximum at 340 nm for gel I and at 342 nm for gel II,²⁰ indicating monomeric naphthalene moieties. Although the lack of a significant excimer peak of naphthalene (about 450 nm²¹) excludes the strong Π-Π interactions between naphthalene groups in gels I and II, the small broad shoulders above 400 nm indicate weak Π-Π interactions between the phenyl and naphthyl groups. This result also agrees with the crystal structure of Nap-FF.²⁰

### Example 6

### Molecular Arrangements

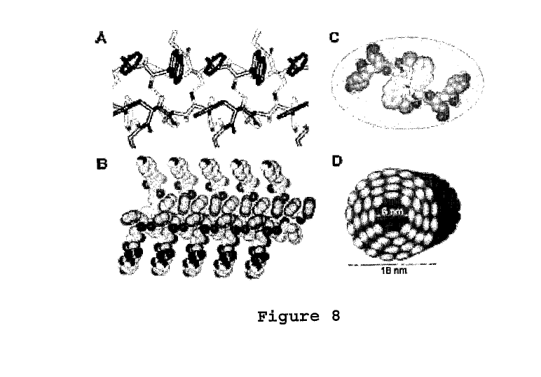

On the basis of the TEM, CD, and fluorescence spectra of the hydrogel of 1 and the X-ray structure of Nap-FF, the present invention proposes the molecular arrangement of 1 in the nanotubes: the hydrogen bonds and hydrophobic interactions (FIG. 8A) cooperatively induce the self-assembly of 1 to yield supramolecular polymers, whose molecular packing (FIG. 8B) exposes the donors and acceptors of the hydrogen bonds that originate from Glu-Tyr fragments (FIG. 8C) and favors their further aggregation to form nanotubes (FIG. 8D).

Although it is impossible to rule out entirely other modes of molecular packing, the superstructure depicted in FIG. 8 is the most probable one because it conforms to the structures of the NAP-FF segment, complies with the CD and emission spectra of 1 in hydrogels, and allows Glu-Tyr groups to be accessible by the enzymes.

### Example 7

### Biocompatibility of the Hydrogelator and Enzymatic Hydrogelation in Vivo

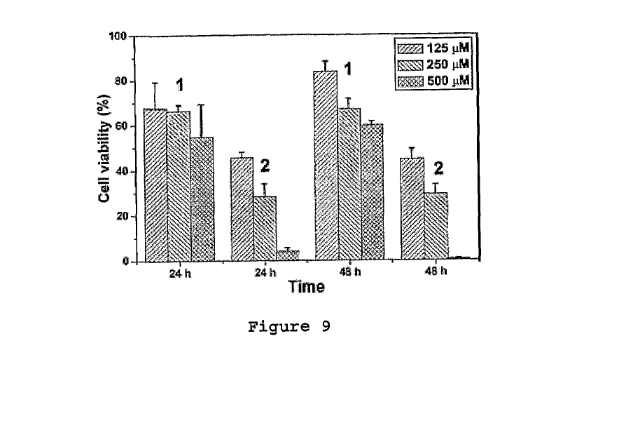

To illustrate the biocompatibility of the hydrogelator, an MTT assay was used to examine the cell viability in the presence of 1 or 2. After 24 h incubation of HeLa cells with 1 and 2, 68 and 46% of the cells survived at 125 μM of 1 and 2, respectively. From the results depicted in FIG. 9, IC50 on the HeLa cell are calculated to be 603 μM for 1 and 93 μM for 2, respectively. Although 2 at high concentration shows an inhibitory effect on the proliferation of the HeLa cells, the hydrogelator (1) is highly biocompatible. The biocompatibility of 1 would be expected to play an important role when the hydrogel is used as material for biomedical applications.

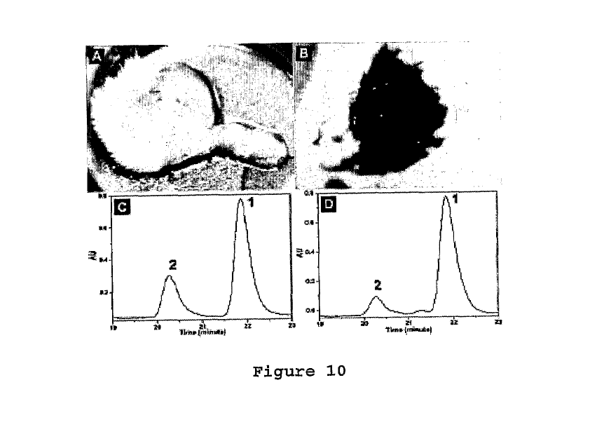

After the preliminary cytotoxicity test confirmed that 1 was biocompatible, the solution of 2 was injected in mice to evaluate the formation of the supramolecular hydrogel of 1 in vivo. Compound 2 (0.5 mL, 0.8 wt %) was injected into each mouse via a subcutaneous mode (i.e., under the skin) and an intraperitoneal mode (i.e., into the abdominal cavity). After 1 h, the hydrogel formed at the location of subcutaneous injection (FIG. 10A). HPLC analysis of the hydrogel reveals that 80.5 (1.2% of 2 turns into 1 (FIG. 10C), which is responsible for the hydrogelation. Although no hydrogel forms in the abdominal cavity, an HPLC test of abdominal fluid indicates that 86.2% of 2 changes back to 1 (FIG. 10D). By comparing HPLC traces in FIG. 10C, D with the HPLC traces of 1 and 2 with known concentrations, the total amount of compounds (both in 2 and 1) that remained in the site of injection were estimated to be relative to the initial amount of 2 injected. For the subcutaneous injection, the value is about 88.4%; for the intraperitoneal injection, the value is about 76.8%. These results suggest that it is crucial to restrict the diffusion of the precursor of the hydrogelator in tissues or organs to ensure the enzymatic hydrogelation in vivo.

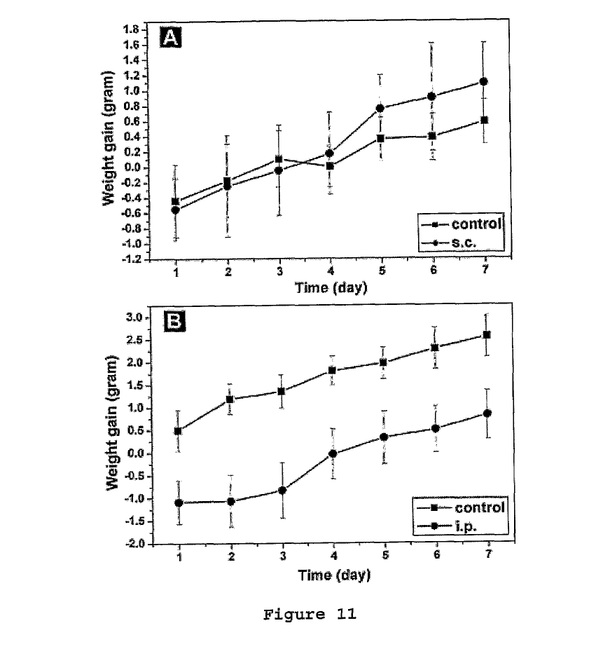

The weight change of the mice was monitored after injecting 2 to assess the in vivo cytotoxicity of 2. As shown in FIG. 11A, the mice that received subcutaneous injection of 2 (0.5 mL, 0.8 wt %) lost body weight (mean=0.55 g, 2.8% decrease) in the first day and so did the mice in the control group (mean=0.44 g, 2.2% decrease). The two groups of mice both started to gain body weight after the second day. The weight loss and gain of the mice in the two groups remained statistically the same, suggesting that subcutaneous administration of 2 at the experimental dosage results in little acute toxicity to the mice. As shown in FIG. 11B, the mice that received intraperitoneal injection of 2 (0.5 mL, 0.8 wt %) lost weight (mean=1.1 g, 5.5% decrease) in the first day whereas the mice in the control group gained weight (mean=0.50 g, 2.5% increase). The two groups of mice, however, had almost the same rate of weight gain (0.34 g/day) from the second day to the seventh day. This result indicates that although the intraperitoneal administration of 2 at the experimental dosage results in acute toxicity in the first day the conversion of 2 to 1 reduces the toxicity remarkably after 24 h. The different responses regarding the injection sites are consistent with the amount of 2 diffused away from the injection sites and the in vitro cytotoxicities of 1 and 2. Being confined in the subcutaneous site, only a small amount of 2 can circulate into the blood and distribute to organs and other tissues, thus lowering the acute toxicity in vivo significantly for subcutaneous injection. Because 2 transforms to 1 eventually, no long-term in vivo toxicity of the hydrogelator is observed for intraperitoneal injection.

### REFERENCES

1. Lodish, H. Molecular Cell Biology, 5th ed.; W. H. Freeman Co.: New York, 2003.
2. Lee, K. Y.; Mooney, D. J. Chem. ReV. 2001, 101, 1869-79. Langer, R. Nature 1998, 392, 5-10. Tang, M. D.; Golden, A. P.; Tien, J. J. Am. Chem. Soc. 2003, 125, 12988-89.
3. Terech, P.; Weiss, R. G. Chem. ReV. 1997, 97, 3133-59. de Loos, M.; van Esch, J.; Stokroos, I.; Kellogg, R. M.; Feringa, B. L. J. Am. Chem. Soc. 1997, 119, 12675-76. Ostuni, E.; Kamaras, P.; Weiss, R. G. Angew. Chem., Int. Ed. 1996, 35, 1324-26. Jung, J. H.; Ono, Y.; Hanabusa, K.; Shinkai, S. J. Am. Chem. Soc. 2000, 122, 5008-09. Shirakawa, M.; Fujita, N.; Shinkai, S. J. Am. Chem. Soc. 2003, 125, 9902-03. Xing, B. G.; Choi, M. F.; Xu, B. Chem Commun. 2002, 362-63. Xing, B. G.; Choi, M. F.; Xu, B. Chem.-Eur. J. 2002, 8, 5028-32.
4. Silva, G. A.; Czeisler, C.; Niece, K. L.; Beniash, E.; Harrington, D.; Kessler, J. A.; Stupp, S. I. Science 2004, 303, 1352-55.
5. Holmes, T. C.; de Lacalle, S.; Su, X.; Liu, G. S.; Rich, A.; Zhang, S. G. Proc. Natl. Acad. Sci. U.S.A. 2000, 97, 6728-33. Zhang, S. G. Nat. Biotechnol. 2003, 21, 1171-78. Niece, K. L.; Hartgerink, J. D.; Donners, J.; Stupp, S. I. J. Am. Chem. Soc. 2003, 125, 7146-47.
6. Behanna, H. A.; Donners, J. J. J. M.; Gordon, A. C.; Stupp, S. I. J. Am. Chem. Soc. 2005, 127, 1193-200.
7. Estroff, L. A.; Hamilton, A. D. Chem. ReV. 2004, 104, 1201-17. de Loos, M.; Feringa, B. L.; van Esch, J. H. Eur. J. Org. Chem. 2005, 3615-31.
8. Sreenivasachary, N.; Lehn, J. M. Proc. Natl. Acad. Sci. U.S.A. 2005, 102, 5938-43. Yoshimura, I.; Miyahara, Y.; Kasagi, N.; Yamane, H.; Ojida, A.; Hamachi, I. J. Am. Chem. Soc. 2004, 126, 12204-05. Hirst, A. R.; Smith, D. K. Chem.-Eur. J. 2005, 11, 5496-508. Estroff, L. A.; Hamilton, A. D. Angew. Chem., Int. Ed. 2000, 39, 3447-50. Menger, F. M.; Caran, K. L. J. Am. Chem. Soc. 2000, 122, 11679-91. Kiyonaka, S.; Sugiyasu, K.; Shinkai, S.; Hamachi, I. J. Am. Chem. Soc. 2002, 124, 10954-55. Bommel, V.; C., K. J.; Stuart, M. C. A.; Feringa, B. L.; Van Esch, J. Org. Biomol. Chem. 2005, 3, 2917-20. Kiyonaka, S.; Sada, K.; Yoshimura, I.; Shinkai, S.; Kato, N.; Hamachi, I. Nat. Mater. 2004, 3, 58-64. Bhuniya, S.; Park, S. M.; Kim, B. H. Org. Lett. 2005, 7, 1741-44. Xing, B. G.; Yu, C. W.; Chow, K. H.; Ho, P. L.; Fu, D. G.; Xu, B. J. Am. Chem. Soc. 2002, 124, 14846-47. Zhang, Y.; Gu, H.; Yang, Z.; Xu, B. J. Am. Chem. Soc. 2003, 125, 13680-81. Zhang, Y.; Yang, Z. M.; Yuan, F.; Gu, H. W.; Gao, P.; Xu, B. J. Am. Chem. Soc. 2004, 126, 15028-29. Yang, Z. M.; Gu, H. W.; Zhang, Y.; Wang, L.; Xu, B. Chem. Commun. 2004, 208-09. Yang, Z. M.; Xu, K. M.; Wang, L.; Gu, H. W.; Wei, H.; Zhang, M. J.; Xu, B. Chem. Commun. 2005, 4414-16.
9. Yang, Z. M.; Xu, B. Chem. Commun. 2004, 2424-25. Yang, Z. M.; Gu, H. W.; Fu, D. G.; Gao, P.; Lam, K. J. K.; Xu, B. AdV. Mater. 2004, 16, 1440-44.
10. Molecular and cellular foundations of biomaterials; Peppas, N. A., Sefton, M. V., Eds.; Elsevier/Academic Press: Amsterdam, 2004.
11. Hu, B.-H.; Messersmith, P. B. J. Am. Chem. Soc. 2003, 125, 14298-99.
12. Kikuchi, K.; Nakamura, K.; Shima, H. Curr. Top. Biochem. Res. 1999, 1, 75-87.
13. Hutton, J. C.; Eisenbarth, G. S. Proc. Natl. Acad. Sci. U.S.A. 2003, 100, 8626-28.
14. Eidenmueller, J.; Fath, T.; Hellwig, A.; Reed, J.; Sontag, E.; Brandt, R. Biochemistry 2000, 39, 13166-75. Yuan, J.; Yankner, B. A. Nature 2000, 407, 802-9.
15. Auch, C. J.; Saha, R. N.; Sheikh, F. G.; Liu, X.; Jacobs, B. L.; Pahan, K. FEBS Lett. 2004, 563, 223-28.
16. den Hertog, J. EMBO Rep. 2003, 4, 1027-31.
17. Kol, N.; Adler-Abramovich, L.; Barlam, D.; Shneck, R. Z.; Gazit, E.; Rousso, I. Nano Lett. 2005, 5, 1343-46. Reches, M.; Gazit, E. Science 2003, 300, 625-27.
18. Yang, Z.; Xu, B. Unpublished results, 2005.
19. Latour, S.; Veillette, A. Curr. Opin. Immunol. 2001, 13, 299-306.
20. Supporting Information
21. Ikeda, H.; Iidaka, Y.; Ueno, A. Org. Lett. 2003, 5, 1625-27.
22. Whitesides, G. M. and Grzybowski, B. Science 295, 2418 (2002).
23. Lehn, J. M. Science 295, 2400 (2002).
24. Stupp, S. I. et al., Science 276, 384 (1997).
25. Love, J. C. L. A. Estroff, J. K. Kriebel, R. G.
26. Nuzzo and Whitesides, G. M., Chem. Rev. 105, 1103 (2005).
27. Nam J. M., Thaxton C. S.,and Mirkin, C. A. Science 301, 1884 (2003).
28. Gu, H., Ho, P.-L., Tsang, K. W., Wang, L., and Xu, B., J. Am. Chem. Soc. 125, 15702 (2003).
29. Hartgerink J. D., Beniash E., Stupp S. I., Science 294, 1684 (2001).
30. Silva, G. A. et al., Science 303, 1352 (2004).
31. Holmes, T. C. et al., P Natl Acad Sci USA 97, 6728 (2000).
32. Kisiday, J. et al., P Natl Acad Sci USA 99, 9996 (2002).
33. Zhang, S. G., Marini, D. M., Hwang, W. and Santoso, S. Curr Opin Chem Biol 6, 865 (2002).
34. Kiyonaka, S. et al., Nat Mater 3, 58 (2004).
35. Yoshimura, I. et al., J Am Chem Soc 126, 12204 (2004).
36. Yang, Z. M. and Xu, B. Chem Commun, 2424 (2004).
37. Xing, B. G. et al., J Am Chem Soc 124, 14846 (2002).
38. Yang, Z. M. et al., Adv Mater 16, 1440 (2004).
39. Gazit, E. Faseb J 16, 77 (2002).
40. Reches, M. and Gazit, E. Science 300, 625 (2003).

1-25. (canceled)

26. A method of making intracellular artificial nanostructures in cell or microorganisms, comprising the steps of:
a. Contacting a properly designed precursor, which does not self-assemble extracellularly, with said cells or microorganisms under conditions permitting the entrance of the precursor into said cells or microorganisms, and
b. Placing the cells or microorganisms under conditions permitting the precursor to convert into nanostructures by hydrogelation or enzymatic reaction, wherein the enzymatic reaction is dephosphorylation, hydrolysis or bond formation,
wherein the nanostructures aggregate inside said cells or microorganisms.

27. A method of making artificial nanostructures inside tissues or organs of an organism, comprising the steps of:
c. Introducing a properly designed precursor, which does not self-assemble extracellularly, into said tissues or organs, and
d. Placing said tissues and organs under conditions permitting the precursor to convert into nanostructures by hydrogelation or enzymatic reaction, wherein the enzymatic reaction is dephosphorylation, hydrolysis or bond formation,
wherein the nanostructures aggregate inside said tissues or organs.

28. The nanostructures made by the method of claim 26 wherein said cells or microorganisms are inhibited or killed by the nanostructures.

29. The nanostructures made by the method of claim 26, wherein the fate of the cells or microorganisms is controlled by the formation of the nanostructures.

30. The method of claim 26 wherein said cells or microorganisms are selectively inhibited or killed for treating multi-drug resistance in cancer or antimicrobial drug resistance in infections.

31. The method of claim 27, wherein the differentiation of cells is stopped.

32. The method of claim 27, wherein the making of said artificial nanostructures inside tissues or organs provides controlled drug release, formation of new therapeutic agents, or in-situ formation of hydrogels to treat chronic diseases.

33. The method of claim 32, wherein the chronic disease is osteoarthritis.

34. A properly designed precursor as used in the method of claim 26, comprising:
e. a hydrophobic group for providing the hydrophobic force to enhance the self-assembly in aqueous environment;
f. a hydrophilic group for providing the major building blocks for self-assembly besides acting as hydrogen bonds acceptors and donors to interact with water; and
g. a cleavable group which is cleavable by an enzyme to serve as an enzymatic switch for tailoring the overall balance of the hydrophobic and hydrophilic interactions and to prevent the hydrogelation of the precursor without enzymatic hydrolysis.

35. The precursor of claim 34, wherein the hydrophobic group is naphthylCH₂—, naphthyl, aromatic, or linear or branched alkyl, or a therapeutic, wherein the therapeutic may be taxol.

36. The precursor of claim 34, wherein the hydrophilic group is phe-phe, a di-, tri-, tetra-, penta, or oligopeptide soluble in water, a carboxylate, an aminoglycoside, an antibiotic, or a water-soluble therapeutic.

37. The precursor of claim 34, wherein the cleavable group is butyric dicarboxylate acid, phosphate, butryic acid, sulfate, ammonium, or ethylene glycol.

38. A composition comprising the precursor of claim 34 and a suitable carrier.

39. A method for treating a subject with cancer, diabetes, Alzheimer's disease, or multiple sclerosis, osteoarthritis, comprising administering to said subject the composition of claim 38 comprising an effective amount of said precursor.

40. The method of claim 39 wherein the precursor is a nap FFGEY type precursor.

41. A kit containing a compartment with the composition of claim 38.
